(19) United States
(12) Patent Application Publication
(10) Pub. No.: US 2012/0060851 A1
(43) Pub. Date: Mar. 15, 2012

(54) Method for Treating Stress Related Disorders

(76) Inventor: Eric C. Amberg, Ann Arbor, MI (US)

(21) Appl. No.: 13/232,054

(22) Filed: Sep. 14, 2011

Related U.S. Application Data
(60) Provisional application No. 61/382,589, filed on Sep. 14, 2010.

(51) Int. Cl. A61B 10/00; G09B 19/00

(52) U.S. Cl. 128/898; 434/219

(57) ABSTRACT

This invention provides a method of assessing and treating stress related disorders, such as PTSD, as physical disorders whose psychological components are the result of conditioning. The method involves both physical testing in order to identify the physical problems underlying the condition and indirect testing to identify and reverse the psychological matrix created by conditioning and the cascading effects of trauma.

### CROSS REFERENCE TO RELATED APPLICATIONS

This application asserts priority from U.S. Provisional Patent Application No. 61/382,589 filed Sep. 14, 2010. Applicant hereby incorporates the disclosures of this application by reference in its entirety.

### FIELD OF THE INVENTION

This invention relates to a method of diagnosing, assessing and treating brain injury, post traumatic stress, post traumatic cranial irritability, and pain resulting from traumatic injuries. Traumatic injuries may involve physical trauma, psychological trauma, or a combination of both.

### BACKGROUND OF THE INVENTION

Both physical trauma and psychological trauma, or combinations thereof can lead to immediate damage and to damage which becomes apparent after the immediate medical problems have been addressed. This damage can lead to a variety of problems such as chronic pain, seizures, mood disorders, problems regulating feelings, which can result in extreme suicidal thoughts, anger, and despair. The diagnosis and assessment of these conditions can be difficult because the presenting symptoms are not associated with a traumatic experience, and the patient may be unaware of the association. A particularly severe form of this type of disorder is called post traumatic stress disorder (PTSD) which is widely observed in combat veterans. Elevated scores on the Iowa Interview for Partial Seizure-like Symptoms (IIPSS) have been correlated with a diagnosis of PTSD (Vincent Roca and Thomas Freeman The Journal of Neuropsychiatry and Clinical Neurosciences 2002; 14:185-189).

### SUMMARY OF THE INVENTION

This invention provides a method of assessing and treating stress related disorders, such as PTSD, as physical disorders whose psychological components are the result of conditioning. The method involves both physical testing in order to identify the physical problems underlying the condition and psychological testing to identify and address the psychological problems created by conditioning and the cascading effects of trauma.

### BRIEF DESCRIPTION OF THE DRAWINGS

FIG. 1 is a flow chart of the regular treatment during the first quartile.

FIG. 2 shows an initial screening test for suicide risk.

### DETAILED DESCRIPTION OF THE INVENTION

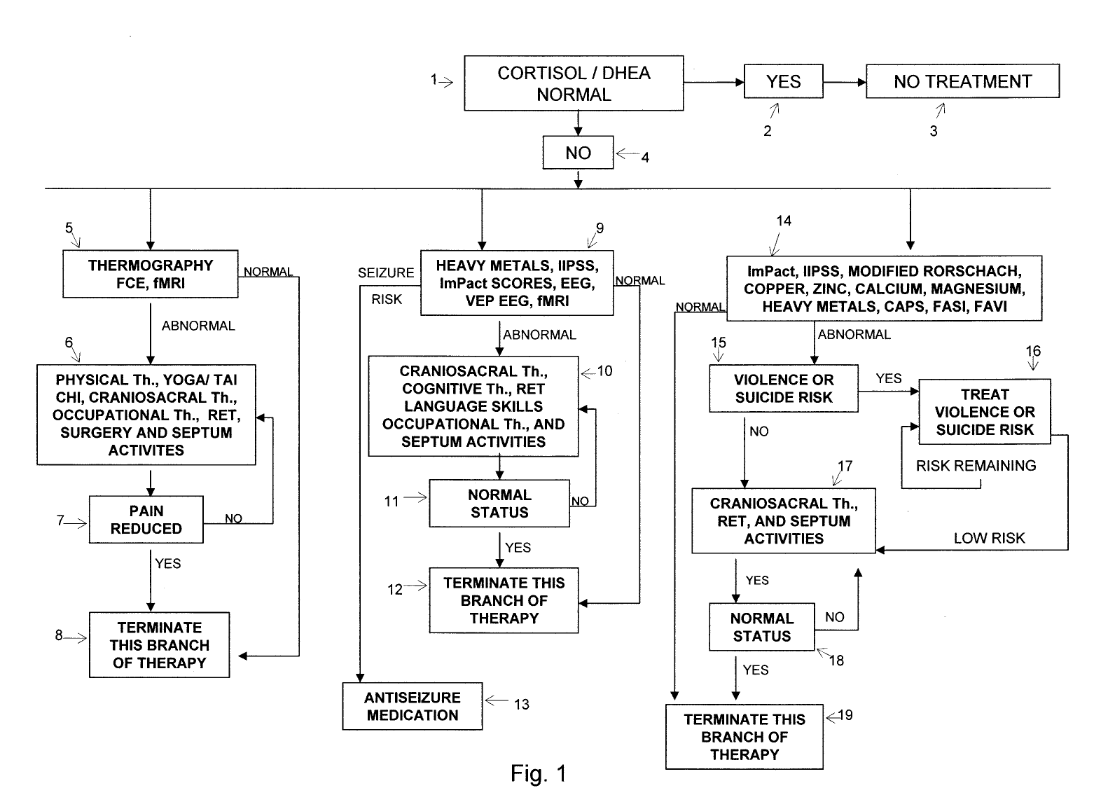

FIG. 1 shows the battery of tests and treatments employed in the method of the present invention. In FIG. 1 the word “therapy” is abbreviated as “Th.” The first regular test performed (1) is a test for the cortisol/DHEA ratio. If this ratio is normal or low (2), treatment is not undertaken (3). If the result is elevated (4) further tests are undertaken.

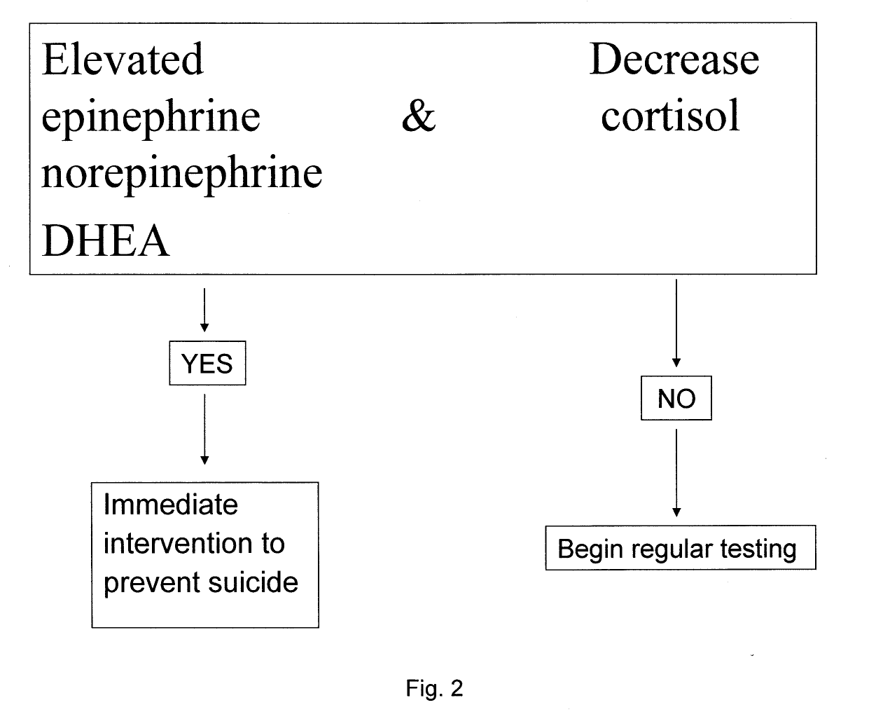

FIG. 2 shows an initial screen for suicide risk. If a pattern of elevated epinephrine, norepinephrine, and DHEA with decrease in cortisol, is found, steps are immediately taken to prevent suicide. If this pattern is not found regular testing is begun as illustrated in FIG. 1. If the pattern of elevated epinephrine, norepinephrine, and DHEA with decrease in cortisol, was initially present, when the epinephrine and norepinephrine subside, regular testing beginning with cortisol and DHEA as shown in FIG. 1 are undertaken.

The tests related to pain (5) are thermography, Functional Capacity Evaluation (FCE), and fMRI. If these tests are normal treatment of pain is not undertaken (8). If pain treatment is indicated, the treatments for pain (6) are selected from the group consisting of physical therapy, occupational therapy, craniosacral therapy, Rational Emotive Therapy (RET), Septum activities, yoga, tai chi, and orthopedic treatments to determine whether surgery is warranted. The therapies may include one or more or even all of these therapies. Pain level is checked (7). If pain persists further therapy (6) is undertaken (6). If pain is sufficiently reduced this branch of treatment is terminated (8).

The tests related to brain function (9) are tests for heavy metals, Post-Concussion Assessment and Cognitive Testing (ImPact) scores, electroencephalogram (EEG), EEG test with visual stimulation (VEP EEG), the Iowa Interview for Partial Seizure-like Symptoms (TIPSS), and functional magnetic resonance imaging (fMRI). If these tests are normal, treatment is not undertaken (12). If these tests indicate a seizure risk, anti-seizure medication is given (13). If the tests are abnormal, but do not indicate a seizure disorder, the indicated therapies (10) are undertaken. The therapies are selected from the group consisting of craniosacral therapy, cognitive therapy, Rational Emotive Therapy (RET), language skill training, and Septum Activities. The therapies are not mutually exclusive, and one or more or even all of these therapies may be undertaken. The patient status is tested (11). If it is not normal, further therapy (10) is undertaken. If it is normal, this branch of therapy is terminated (12).

The tests relating to degree to which brain irritability is manifested by the symptoms of PTSD (14) include the Iowa Interview for Partial Seizure-like Symptoms (IIPSS), the urine levels of calcium, magnesium, zinc, and copper, urine levels of heavy metals, Clinician Administered PTSD Scales (CAPS), FASI, FAVI, ImPact, and a modified Rorschach test. If the results of these tests are normal, this branch of therapy is terminated (19). If the results show abnormalities, the risk of violence or suicide is determined (15). If there is a risk of violence or suicide, this is treated (16). When the risk is reduced, the patient is treated with therapies selected from craniosacral therapy, Rational Emotive Therapy (RET), and septum activities (17). The therapies are not mutually exclusive, and one or more or even all of these therapies may be undertaken. When normal status is restored (18), this branch of therapy is terminated (19).

There are a wide variety of stress related disorders of varying degree of severity. Symptoms may range from irritability and mild insomnia to extremely debilitating conditions. Post traumatic stress disorder (PTSD) is a particularly severe stress related disorder. PTSD can be disabling and prevent an individual from participating in normal activities such as maintaining a job or engaging in normal family activities. Although the stress related disorders are diverse, and the symptoms may appear to be unrelated, traumatic cranial irritability is deeply related and may be the underlying cause of all stress related disorders. Because of stress, brain activity in the stress related regions of the brain has been increased leading to traumatic cranial irritability. Accordingly, the present invention treats the stress related disorders as physical ailments which are treated by reducing activity in the stress related regions of the brain and restoring brain activity to a normal balance.

Traumatic cranial irritability is found in individuals who have experienced traumatic events and injuries. It is more common among those who experience repeated stress as part of their daily life. Thus this condition is found in policemen and firemen who have responded to particularly troubling disasters. However, the highest frequency of traumatic cranial irritability is found in combat veterans, and PSTD is frequently identified in these individuals.

The high frequency of traumatic cranial irritability among combat veterans relates to the repeated prolonged stress of combat. In addition, the training given to prepare soldiers for combat duty predisposes them to develop traumatic cranial irritability. One element of training which can predispose the combat soldier to traumatic cranial irritability is the heightened sensitivity to potential danger which a combat soldier must have in order to survive. When a soldier is on a combat patrol everything must be regarded with suspicion. Things which appear ordinary in the civilian world, such as a group of people on a street or a small mound of dirt could be dangerous to the soldier. The people could be enemy combatants, and the mound of dirt could be an explosive device. Perhaps the mound of dirt is a decoy to induce the soldier to walk away from the dirt over a more skillfully buried land mine. Another element of training is the ability to overlook things like hunger, the need for sleep, cold and heat. These elements of training are achieved through heightening the function of the amygdala, a brain center related to survival, action, and balance. Other brain centers may also be activated and others suppressed. This altered brain activity is useful in combat, and even necessary for survival, but is maladaptive in civilian society.

Unfortunately, with training and prolonged stress, the altered brain activity becomes chronic and is not reduced even when the combat soldier is discharged. Once in the civilian world the dangers of combat are no longer present. However, the soldier's heightened awareness and suspicion remain. Ordinary things still create stress even though they are unlikely to be dangerous. Some people are able to quiet their heightened awareness and return to civilian life. However, some cannot, and they become conditioned to dealing with stresses that those around them do not feel. Even worse, they have to deal with stresses that they know are irrational. In the civilian world, the mound of earth is not a bomb, and the combat veteran knows that. However, the stress response remains. The altered brain activity, and the stress which that creates, become conditioning events leading to the emotional disturbances of the traumatic cranial irritability.

Although traumatic cranial irritability is most frequently observed in combat veterans the same mechanism is at play in others who suffer from stress related disorders. Accordingly, the same treatment methods can be used with all people suffering from stress disorders. However, because of the large number of combat veterans having stress related disorders, it is anticipated that the majority of the people to be treated by the present method would be combat veterans.

Although psychological problems are often the presenting symptoms, traumatic cranial irritability is a physical disorder relating to altered brain activity. The psychological problems are the result of conditioning, and may be considered to be an adaption to cope with the altered brain activity. The first step in treating traumatic cranial irritability is to identify the underlying physical problems relating to brain activity, and the psychological problems which have resulted from the altered brain activity.

Since training caused the combat soldier to develop the altered brain activity necessary for combat, the present method focuses on training to restore normal brain activity. The goal is to reduce the activity level of the amygdala and increase the activity of the septum regions of the brain and to be able to assess these changes internally. Patients will have access to the assessment tools, described below, at any time to assess the strength of their choices. Other less measurable aspects of the program include family realignment, spiritual sessions and other types of personal quiet time. These aspects of the program take place in the second, third, and fourth quartiles of the treatment program. As a group they are referred to as restoration of normal activities.

At various times throughout the patient's participation in the training program there will be opportunities for evaluation of the patient's progress in resolving the presenting symptoms. Over time, it is anticipated that there will be changes and improvements in the symptoms. The progress may not be uniform, and some symptoms may improve more quickly than others. As the symptoms change there will be changes in orientation and treatment strategies.

The method of treatment begins with initial screening and a battery of tests. The initial screening is to find out if there is a risk of suicide. It has been found that in patients who suffer from PSTD and are highly likely to commit suicide, there are profound alterations in stress hormones: elevated epinephrine and norepinephrine and elevated DHEA with decrease in cortisol. Accordingly, the initial step is a suicide screen consisting of testing for elevated epinephrine and norepinephrine and elevated DHEA with decrease in cortisol. This is shown in FIG. 2. If this pattern of elevated epinephrine, norepinephrine, and DHEA with decrease in cortisol, is found, steps are immediately taken to prevent suicide. If this pattern is not found regular testing is begun as illustrated in FIG. 1. If the pattern of elevated epinephrine, norepinephrine, and DHEA with decrease in cortisol, was initially present, when the epinephrine and norepinephrine subside, regular testing beginning with cortisol and DHEA as shown in FIG. 1 are undertaken. Special notice of association of DHEA with a risk of self injurious behavior is taken.

The first regular tests are the determination of serum levels of cortisol, a steroid hormone associated with acute anxiety, DHEA (5-Dehydroepiandrosterone, IUPAC name 3S,8R,9S,10R,13S,14S)-3-hydroxy-10,13-dimethyl-1,2,3,4,7,8,9,11,12,14,15,16-dodecahydrocyclopenta[a]phenanthren-17-one), a precursor to cortisol. High ratios of cortisol to DHEA correlate with pain, stress, and clinical depression. If the ratio is normal, no further treatment is undertaken. If the ratio is elevated, further test are done. The further tests are designed to evaluate three areas of concern. The first group of tests relates to the degree of pain which the patient is experiencing. The second group of tests relates to the level of brain or nervous system damage may be present. The third group associated with acute and chronic anxiety relates relates to degree to which brain irritability is manifested by the symptoms of PTSD and seizure disorders.

The tests relating to pain include Functional capacity evaluation (FCE), fMRI, and thermography. Functional capacity evaluation (FCE) is used to evaluate the differences between limbs and the impairment of motion due to pain. The FCE test involves a series of light exercises that included toe-touching, carrying a ten pound weight, crawling, walking, sitting, and other activities appropriate for the patient's condition. For example patients reporting shoulder pain would have test components relating to shoulder motion while patients reporting leg pain would have test components relating to leg motion. Thermography is used to assess body heat levels. The thermographic technique forms images of the body using infrared radiation radiated by the body instead of visible light reflected from the body. Pain is often associated with inflammation, and the inflammatory process causes the inflamed area of the body to radiate more heat, which can be detected by thermography. FMRI helps to determine whether structural injuries a present for which more invasive procedures may be warranted. If there are abnormalities in the test results the patient receives physical therapy, yoga, tai chi, craniosacraal therapy, Rational Emotive Therapy (RET), occupational therapy, and Septum Activities. When pain is reduced to an acceptable level this branch of therapy is terminated.

The tests relating to brain or nervous system disorder included include Post-Concussion Assessment and Cognitive Testing (ImPact) scores, electroencephalogram (EEG), EEG test with visual stimulation (VEP EEG), the Iowa Interview for Partial Seizure-like Symptoms (IIPSS), and functional magnetic resonance imaging (fMRI). Post-Concussion Assessment and Cognitive Testing (ImPACT) is used if cortical injuries are suspected. ImPACT is a widely used, computerized concussion evaluation system. Patients having below normal ImPACT scores, which indicate cognitive impairment due to cortical injuries, are treated for such injuries, before other treatment is undertaken. An electroencephalogram (EEG) and the visually evoked potential EEG tests are performed to assess the possibility of a seizure disorder. The EEG test does not respond to individual neurons. Instead it responds to the synchronized activity of a network of neurons. The VEP EEG is an EEG measured while the patient receives stimulation of the visual field. Seizure disorders are indicated by high intensity waves at higher than normal frequency. If there is a risk of seizures, anti-seizure medications are given. Depending upon the cause of the potential seizures, anti-seizure medication may be required on a long term basis. For example, if the seizures are the result of a brain lesion, which cannot be treated, anti-seizure medication may be required for more long term usage. The battery of tests also includes neurological studies of the brain including functional magnetic resonance imaging (fMRI), and the Iowa Interview for Partial Seizure-like Symptoms (IIPSS) to further assess for limbic disorder.

The tests relating to degree to which brain irritability is manifested by the symptoms of PTSD include the Iowa Interview for Partial Seizure-like Symptoms (IIPSS), the urine levels of calcium, magnesium, zinc, and copper, urine levels of heavy metals, Clinician Administered PTSD Scales (CAPS), FASI, FAVI, ImPact, and a modified Rorschach test. Abnormal urine levels of calcium, magnesium, zinc, and copper are associated with limbic disorders. Abnormal urine levels of heavy metals are associated with brain disorders. An elevated ratio of copper to other metals, especially calcium, relate to seizure disorders, prolonged stress, and consequential behavioral disorders. The tests also include a PTSD questionnaire e.g. Clinician Administered PTSD Scales (CAPS), FASI, and FAVI (two paper and pencil tests designed to measure self destructiveness and the potential for violence), ImPact, and a modified Rorschach test to assess for implosiveness, that is self directed violence, and explosiveness, that is outward directed violence. The modified Rorschach test is scored using a system developed by Bruno Klopfer. There are several scales used in scoring the test. The M scale relates to the subject's ability to recognize human form as opposed to less stable responses. It is also noted how the subject handles color and form. Some subjects relate to both color and form (CF) while other subjects relate to color only (C). In extreme cases, the subject's responses may not relate to color or form. In responses relating to color and form, it is important to note the level of violence in the description of the images. Some subjects report violent images such as bombs and volcanoes, while others report deteriorating or implosive conditions such as dry leaves or earthquakes.

If such violent tendencies are found, they receive immediate attention and are initially treated with sedatives. This is followed by exploratory work to uncover possible psychogenic and/or physiogenic sources of the disturbance. Once the triggers are discovered, supportive therapy is initiated to reassure the patient that his condition will be monitored and appropriately addressed. Cortisol levels are determined until the patient's condition has stabilized. Dydactic interaction is essential for the patient to recognize that the condition is not mental and that it can be improved by using corrective measures. Once the risk of violence or suicide is averted, therapies include craniosacral therapy, and septum activities.

The present method focuses on training to restore normal brain activity. The goal is to reduce the activity level of the amygdala and increase the activity of the septum regions of the brain. There are several treatment options. One treatment option is Rational Emotive Therapy (RET). This therapy is based on the premise that behavior is learned and can be un-learned. RET involves training the patient to think about things in a different manner. Changing the way a person thinks can change the way he feels. For example, if a patient suffers from fear or anxiety in certain situations, RET does not focus on the cause of the fear or anxiety, but instead trains the patient to respond to the situation in a different, calmer manner. Every person has a framework, or approach, to processing information. In patients with stress related disorders this framework or approach is maladaptive for their current situation and cognitive therapy focuses on changing the patient's framework or approach. The patient must be taught to identify negative thoughts and learn how to replace these negative thoughts with more positive thoughts. In doing this, the therapy reduces the activity the stress associated brain centers such as the amygdala and increases the activity of other regions of the brain. As brain activity is brought into more normal balance, the behavioral symptoms of the traumatic cranial irritability are ameliorated.

A second treatment option is cognitive therapy. It is one of the options used to address cortical damage. The method is both computer based, and conducted by a therapist. Using individual software, specific areas of impairment are targeted. The patient using the software is provided with specific computer exercises to work on. The therapist is available to provide assistance, but the patient assumes the primary role in restoring his functioning. Because the patient is in charge, he/she can monitor progress and difficulties and use assessment tools to monitor progress and to determine the status of his condition.

A third treatment option is physical therapy. These strategies involve yoga, tai chi, exercise therapy, craniosacral therapy, and other methods known to down regulate the nervous system. Modified craniosacral therapy may be used to help the patient quiet the nervous system. Craniosacral therapy includes modified breathing activities with visualizations to create greater self awareness of bodily conditions and steps required for self correction and modification. The effect of yoga, relaxation exercises and reading may be compared to the effect of more active strategies such as basketball, use of the fitness path or Wii system to determine which modality produces greater septum activity (as measured by endorphin, oxytocin and enkeplhalin levels) and under what conditions. These septum activities are important because they increase the activity of the septum region of the brain, while decreasing the activity of the amygdala. For example, game playing may have better results in the morning and early afternoon, while more focused, less active activities later in the evening.

Similarly, diet, vocational activities and library research, also enhance septum functioning, while action movies can lead to greater amygdala activity. It is important for the patient to recognize these distinctions and learn how to act accordingly.

Vocational training is a fourth treatment option. The vocational training is designed to provide the patient a productive activity which can replace the stress related activities. If the patient has a vocation which is satisfactory, the training may be directed toward the development of a hobby which could be pursued independently of work. Involvement in a job or other activity helps the patient integrate into a more normal setting and thereby helps to bring brain activity back to a more normal pattern. The particular training selected is derived from the biography along with tests of interests and aptitudes. Obviously, training for which the patient has no aptitude, or no interest, does not serve as a proper treatment for traumatic cranial irritability.

Occupational therapy is a fifth treatment option. Occupational therapy is patient-centered therapy in which the therapist first develops an understanding of the patient's primary roles and occupations. The therapist then determines the patient factors that affect the patient's ability to engage and fully participate in these activities. The factors to be considered include social-emotional skills, cognitive processes, and motivational issues. The therapist then helps the patient understand and develop the performance skills needed to perform the patient's roles and occupations.

Although the teaching of language skills is not generally considered to be a treatment method, it may be used as part of the program. Many patients do not read well or express themselves well. This lack of skill can be stressful because it can limit the employability of the patient, and/or of the patient to fully integrate within personal and extended communities. Accordingly, the later phases of treatment which focus on restoration of normal activities are aided by improving the patient's language skills

The overall program is divided into quartiles, which provide different levels of care. The program is not an out patient program. Instead, the patients reside at the treatment facility until they are discharged. In the 1st quartile patients remain within the program without any passes permitted. In the 2nd quartile patients may be given the opportunity to have monitored passes in the nearby town. In the 3rd quartile patients have the opportunity to travel into town without escort. In the 4^{th} quartile patients have overnight passes and prepare for discharge.

During the first quartile the method focuses on assessment of the patient's condition and specific therapies aimed at the underlying causes of brain irritability. The initial step is a suicide screen consisting of testing for elevated epinephrine and norepinephrine and elevated DHEA with decrease in cortisol. This is shown in FIG. 2. If this pattern of elevated epinephrine, norepinephrine, and DHEA with decrease in cortisol, is found, steps are immediately taken to prevent suicide. If this pattern is not found regular testing is begun as illustrated in FIG. 1. If this pattern is not found regular testing is begun as illustrated in FIG. 1. If the pattern of elevated epinephrine, norepinephrine, and DHEA with decrease in cortisol, was initially present, when the epinephrine and norepinephrine subside, regular testing beginning with cortisol and DHEA as shown in FIG. 1 are undertaken. Special notice of association of DHEA with a risk of self injurious behavior is taken.

FIG. 1 is a flow chart showing the process of regular treatment in the first quartile. The assessment of the patient's condition begins with a tests for cortisol and DHEA. If the cortisol and ratio between cortisol and DHEA are normal, the patient's problems are not stress related and stress related treatment is not undertaken. In the next step of the treatment method, the patient undergoes three groups of tests. The groups could be done sequentially, but it is preferable to do all three groups together. The first group of tests relate to pain. This group includes thermography and functional capacity evaluation (FCE). If there are abnormalities in the test results the patient receives physical therapy, yoga, exercise therapy, and craniosacral therapy. When pain is reduced to an acceptable level this branch of therapy is terminated.

In the second, third and fourth quartile treatment involve the gradual restoration of normal activities with the goal of terminating therapy and discharging the patient. The therapies used during the first quartile may continue in the second through fourth quartiles. However, the goal is to create a permanent change in the patient, so that therapy can end and the patient can resume a normal life. In the second quartile the patient will have meetings with his family, supervised outings and vocational guidance. In the third quartile the patient may have overnight visits with his family, semi-independent outings and possibly some independent outings. The fourth quartile is devoted to discharge planning and making arrangements for independent living. The second, third, and fourth quartiles of the program are less structured than the first quartile and are adjusted to fit the specific needs of the patient. For example, if the patient has a supportive family, emphasis on family relationships would be important. On the other hand, if the patient has no particular family ties, emphasis would be placed on relationships with other social groups.

I claim:

1. A method of treating a stress related disorder comprising the steps of:
(a) performing diagnostic tests relating to stress and anxiety selected from the group consisting of measuring the serum epinephrine level, the serum norepinephrine level, serum cortisol level, measuring the serum DHEA level, thermography or functional capacity evaluation (FCE);
(b) performing diagnostic tests relating to brain function selected from the group consisting of measuring urine levels of heavy metals, administering the ImPACT test, conducting an electroencephalogram (EEG), conducting an electroencephalogram with visual stimulation (VEP EEG), or conducting functional magnetic resonance imaging (fMRI);
(c) performing diagnostic tests relating to post traumatic stress disorder (PTSD) selected from the group consisting of administering the Iowa Interview for Partial Seizure-like Symptoms (IIPSS), administering the ImPact test, administering the modified Rorschach ink blot test, measuring urine levels of copper, zinc, calcium, and magnesium, administering the CAPS test, administering the FASI test, or administering the FAVI test;
(d) administering treatments selected from the group consisting of Rational Emotive Therapy, cognitive therapy, physical therapy, yoga, tai chi, exercise therapy, craniosacral therapy, relaxation therapy, occupational therapy, vocational training, and surgery;
(e) administering treatments to restore normal activities.

2. A method according to claim 1 in which the treatments are selected from the group consisting of Rational Emotive Therapy, physical therapy, exercise therapy, occupational therapy, and vocational training,

3. A method according to claim 1 in which the treatments are selected from the group consisting of cognitive therapy, physical therapy, exercise therapy, occupational therapy, and vocational training,

4. A method according to claim 1 in which the treatments are selected from the group consisting of Rational Emotive Therapy, yoga, tai chi, exercise therapy, occupational therapy, and vocational training,

5. A method according to claim 1 in which the treatments are selected from the group consisting of cognitive therapy, yoga, tai chi, exercise therapy, occupational therapy, and vocational training,

6. A method according to claim 1 in which the treatments are selected from the group consisting of Rational Emotive Therapy, physical therapy, exercise therapy, craniosacral therapy, relaxation therapy, occupational therapy, and vocational training,

7. A method according to claim 1 in which the treatments are selected from the group consisting of cognitive therapy, physical therapy, exercise therapy, craniosacral therapy, relaxation therapy, occupational therapy and vocational training,

8. A method according to claim 1 in which the treatments are selected from the group consisting of Rational Emotive Therapy, cognitive therapy, physical therapy, exercise therapy, craniosacral therapy, relaxation therapy, occupational therapy, and vocational training,

9. A method according to claim 1 in which the treatments are selected from the group consisting of Rational Emotive Therapy, cognitive therapy, physical therapy, yoga, tai chi, craniosacral therapy, relaxation therapy, occupational therapy, and vocational training,

10. A method according to claim 1 in which the treatments are selected from the group consisting of Rational Emotive Therapy, cognitive therapy, physical therapy, exercise therapy, craniosacral therapy, occupational therapy, and vocational training,
